(19) United States
(12) Patent Application Publication
(10) Pub. No.: US 2010/0116658 A1
(43) Pub. Date: May 13, 2010

(54) ANALYSER AND METHOD FOR DETERMINING THE RELATIVE IMPORTANCE OF FRACTIONS OF BIOLOGICAL MIXTURES

(76) Inventors: Tomislav Smuc, Zagreb (HR); Fran Supek, Zagreb (HR)

Correspondence Address: TRASKBRITT, P.C., P.O. BOX 2550, SALT LAKE CITY, UT, 84110, US

(21) Appl. No.: 12/451,714

(22) PCT Filed: May 28, 2008
(86) PCT No.: PCT/HR2008/000019

§ 371 Date: Nov. 24, 2009

(30) Foreign Application Priority Data
May 30, 2007 (HR) PCT/HR2007/000016

(51) Int. Cl. G01N 27/26; C12M 1/34; C12Q 1/02

(52) U.S. Cl. 204/452; 435/288.6; 435/29; 204/461; 204/600

(57) ABSTRACT

An analyser and method for determining the relative importance of fractions of biological mixtures projects data obtained from at least two mixtures with different physiological conditions by chromatographic or mass spectrometric measurement into a second attribute space using a projection technique such as principal component analysis. The projected data is then filtered using a feature selection method such as ReliefF, before being projected back to the first attribute space using a reversion of the projection technique. This back-projected data is then filtered using another feature selection method such as ReliefF before being output in a human-readable form. This technique improves the clarity of the data by removing components relating to noise or systematic error and therefore makes it easier to determine which fractions of biological mixtures are most important for distinguishing between the different biological mixtures and identifying the physiochemical attributes that correspond to the difference in physiological conditions. The technique is useful in medical diagnostics, quality control and basic biomedical science.

The present invention relates to an analyser for determining the relative importance of fractions of biological mixtures, a method of determining the relative importance of fractions of biological mixtures, a computer program comprising instructions which, when executed, cause an analyser to perform the method, a computer-readable medium comprising the computer program and a signal carrying the computer program.

It is well known to separate biological mixtures such as mixtures of proteins in tissue extracts into fractions in order to determine the amount of particular fractions with a certain quality for practical uses including scientific research into the constituents of the mixture or biomedical testing, for example to determine the nature of a tumour. In particular it is known to compare a plurality of different biological mixtures in order to determine the physiochemical properties which cause or indicate the different physiological conditions between the different biological mixtures.

Methods of separation can be mass spectrometric or chromatographic and include but are not limited to: capillary electrophoresis, gel electrophoresis, paper electrophoresis, ion-exchange chromatography, affinity chromatography, gel filtration, partition chromatography, adsorption chromatography and mass spectrometry.

Biological mixtures include but are not limited to: cell culture or tissue extracts of proteins, lipids, saccharides and nucleic acids (RNA and DNA), which may undergo prior purification to enrich the mixture with a single component e.g. all, or a representative of phosphoproteins, glycoproteins, nucleic acids containing certain sequences or nucleotide modifications or bound to certain proteins or prior digestion of mixture components e.g. treatment with proteolytic enzymes or restriction nucleases.

Such separation methods produce a plurality of fractions of the original mixture, each containing biomolecules characterised by a level of a certain physicochemical property. For instance, gel electrophoresis of DNA fragment mixture separates the fragments by length where parts of gel can be considered fractions, and affinity chromatography of proteins produces fractions containing proteins of different binding affinity towards the carrier matrix. The quantity of a certain class of biomolecule in a fraction can be determined by spectrometric measurement of absorbed, reflected or emitted (as in fluorescence) light of one or more wavelengths, measurement of other optical properties including refractivity and polarization of light, and electric properties, including conductivity. The measurements may be preceded by a specific or non-specific staining or radioactive labelling; for instance, a radioactively labelled oligonucleotide probe can be used to specifically detect a DNA fragment of interest in an agarose electrophoresis gel, while an intercalating dye would stain all nucleic acids non-specifically.

However, it is difficult to easily determine from the measurements of two or more different biological mixtures which particular fractions relate to the physiological differences between the different mixtures. This can be due to noise or systematic errors in carrying out the measurements induced by the instruments or the experimental protocol.

Various techniques have been used to reduce noise or otherwise clarify the results of chromatographic or mass spectrographic methods. Chromatograms and complex chromatographic patterns have been processed using different methods: principal component regression analysis (Jellum et al, J Pharm Biomed Analysis 9, (1991), 663-669), applying Fourier transform and principal component regression to rapidly determine individual species in the sample (Cholli et al., U.S. Pat. No. 5,985,120). Improving signal to noise ratio in an electropherograms by binning measured data points into variable size bins and subsequent Fourier filtering is described in Anderson, U.S. Pat. No. 5,098,536. T. G. Stockham and J. T. Ives in U.S. Pat. No. 5,273,632 disclose complex signal processing based on blind deconvolution and homomorphic filtering of electrophoretic signals. Szymanska et al., Journal of Pharmaceutical and Biomedical Analysis 43 (2007) 413-420 teaches applying baseline correction, denoising, selection of a target sample, optimisation of electropherogram alignment, normalisation of obtained results by known creatinine concentrations and, finally PCA analysis to electrophoretic data. Shin and Markey, Journal of Biomedical Informatics 39 (2006) 227-248 is a review of machine learning approaches for use in mass spectrometry data and discusses the components of preprocessing, feature extraction, feature selection, classifier training and evaluation.

However, none of these known techniques can consistently remove all of the noise or systematic errors in the data. Thus there is a technical problem that current techniques result in a of lack of clarity of filtered data which makes determination of the relative importance of fractions of biological mixtures separated by a chromatographic or mass spectrometric method originating from cells or tissues with different physiological conditions difficult or impossible.

The inventive solution to this problem according to the invention comprises an analyser for determining relative importance of fractions in biological mixtures separated by a chromatographic or mass spectrometric method originating from cells or tissues with different physiological conditions, the analyser arranged to:
a. obtain measurements of physiochemical attributes of a plurality of cells or tissues with first and second physiological conditions in the form of a data set in a first attribute space;
b. project the data set into a second attribute space using a projection technique such that the data is described as a plurality of components mathematically constructed from the original data set;
c. filter the data set in the second attribute space using a feature selection method to determine which components of the data set are most relevant for determining the different physiological conditions by comparing for each individual component, the distribution of values for that component relating to the first physiological condition and the distribution of values for that component relating to the second physiological condition and discarding those components where the difference between the distribution of values in respect of the first and second physiological conditions is low, to provide a filtered data set;
d. back-project the filtered data set back to the first attribute space using a reversion of the projection technique used previously at step (b); then
e. filter the back-projected data set in the first attribute space using a feature selection method to determine which attributes of the back-projected data set are most relevant for determining the different physiological conditions by comparing how the distribution of values of each attribute of the data set differs between the first physiological condition and the second physiological condition and discarding those attributes where the difference in distribution of values is low; and
f. output the results of step (e) in a human-readable format such that the physiochemical attributes that correspond to the differences in physiological conditions between the plurality of cells or tissues can be identified.

It has been found that by using an analyser carrying out steps a-f where a feature selection method, such as ReliefF, is carried out in the second attribute space, the removal of components relating to noise and systematic errors is facilitated and the identification of physiochemical attributes that correspond to differences in physiological conditions is improved.

Also provided is a method of determining relative importance of fractions in biological mixtures separated by a chromatographic or mass spectrometric method originating from cells or tissues with different physiological conditions, comprising:
a. obtaining measurements of physiochemical attributes of first cells or tissues with first physiological conditions and second cells or tissues with second physiological conditions, in the form of a first data set in a first attribute space;
b. projecting the data set into a second attribute space using a projection technique such that the projected data set is described as a plurality of components mathematically constructed from the first data set; and characterised by:
c. filtering the data set in the second attribute space using a feature selection method to determine which components of the data set are most relevant for determining the different physiological conditions by comparing for each individual component, the distribution of values for that component relating to the first physiological condition and the distribution of values for that component relating to the second physiological condition and discarding those components where the difference between the distribution of values in respect of the first and second physiological conditions is low, to provide a filtered data set;
d. back-projecting the filtered data set back to the first attribute space using a reversion of the projection technique used previously at step (b) to provide a back-projected data set; then
e. filtering the back-projected data set in the first attribute space using a feature selection method to determine which attributes of the back-projected data set are most relevant for determining the different physiological conditions by comparing how the distribution of values of each attribute of the data set differs between the first physiological condition and the second physiological condition and discarding those attributes where the difference in distribution of values is low; and
f. outputting the results of step (e) in a human-readable format such that the physiochemical attributes that correspond to the differences in physiological conditions between the plurality of cells or tissues can be identified.

As with use of the analyser according to the invention this method of carrying out steps a-f where a feature selection method, such as ReliefF, is carried out in the second attribute space, facilitates the removal of components relating to noise and systematic errors and the identification of physiochemical attributes that correspond to differences in physiological conditions is improved.

Also provided is a computer program comprising instructions which, when executed, cause an analyser to perform the method; a computer-readable medium comprising a computer program; and signal carrying the computer program. All of which share the same advantages as the method and apparatus mentioned above.

By way of a non-limiting example, an embodiment of the invention will now be described with reference to the accompanying drawings in which:

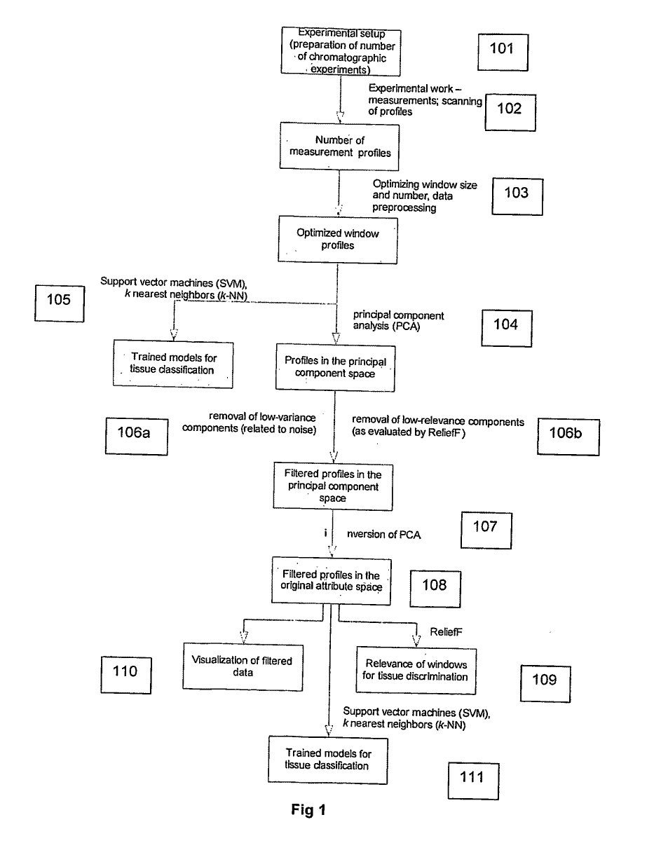

FIG. 1 shows a flow chart of the steps carried out in the embodiment of the invention;

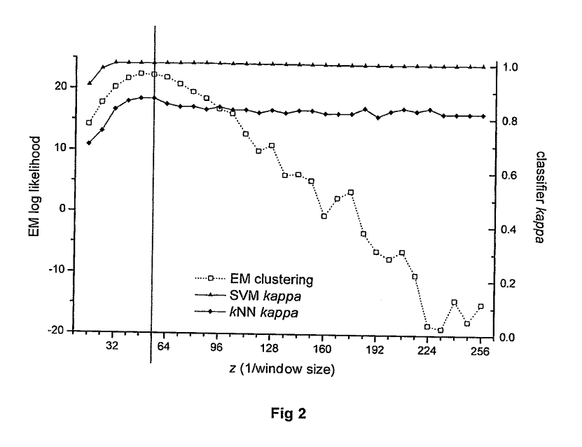

FIG. 2 shows a graph used to determine the optimal window size used in the embodiment of the invention;

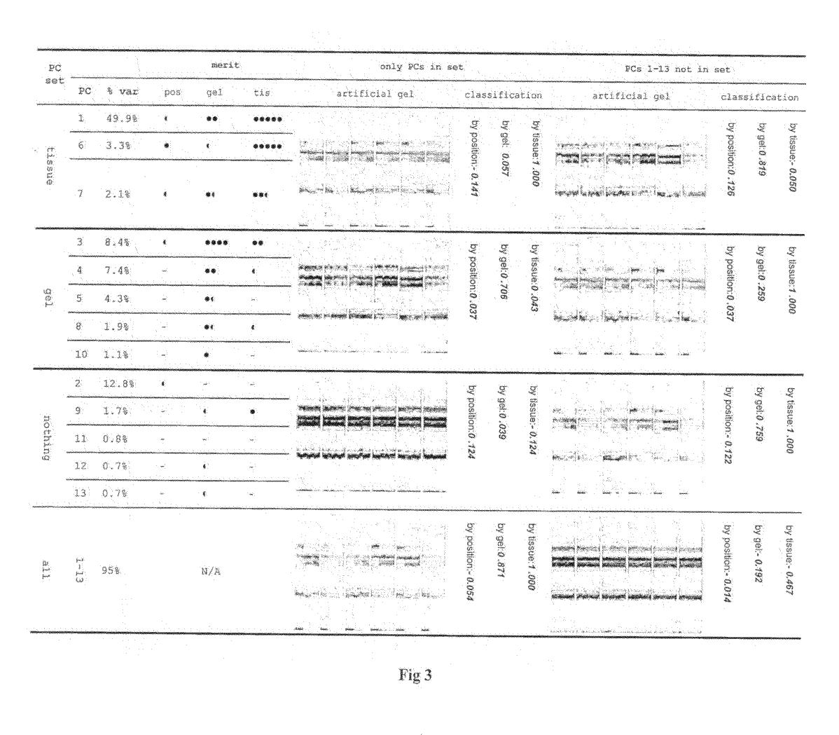

FIG. 3 shows an artificial gel according to the embodiment of the invention together with comparative artificial gels;

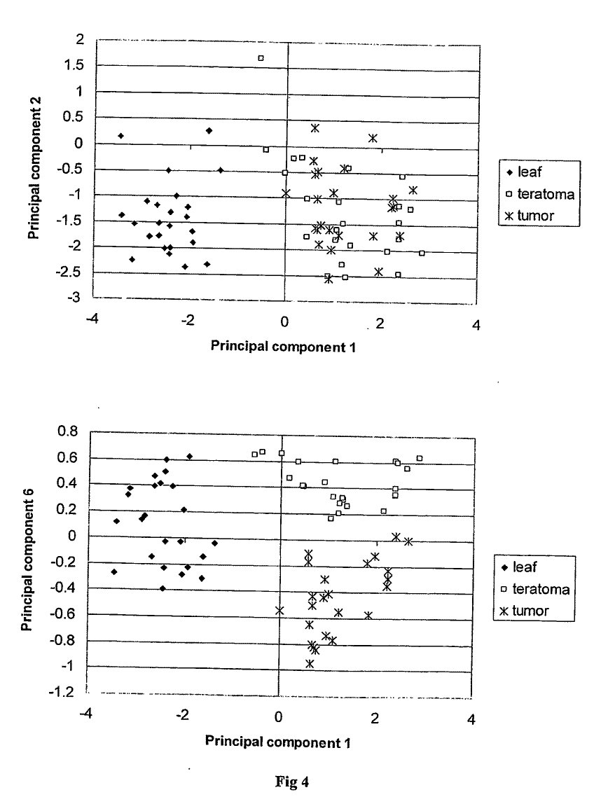

FIG. 4 shows two graphs illustrating the relevance of certain individual principal components determined according to the invention for discrimination according to tissue type;

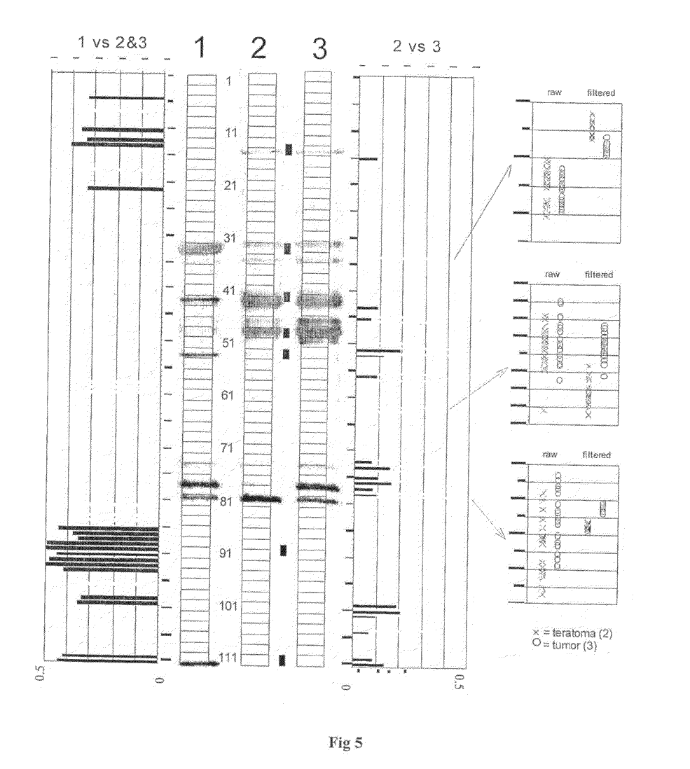

FIG. 5 shows an extract from the gel used in the first embodiment of the invention and graphs showing the ReliefF scores of the data filtered according to the embodiment of the invention together with the ReliefF scores of the raw data as a comparative example;

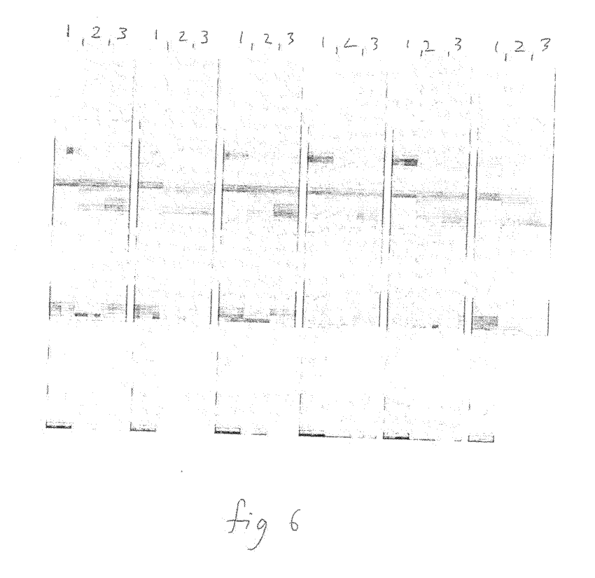

FIG. 6 shows an enlarged view of one of the artificial gels of FIG. 3; and

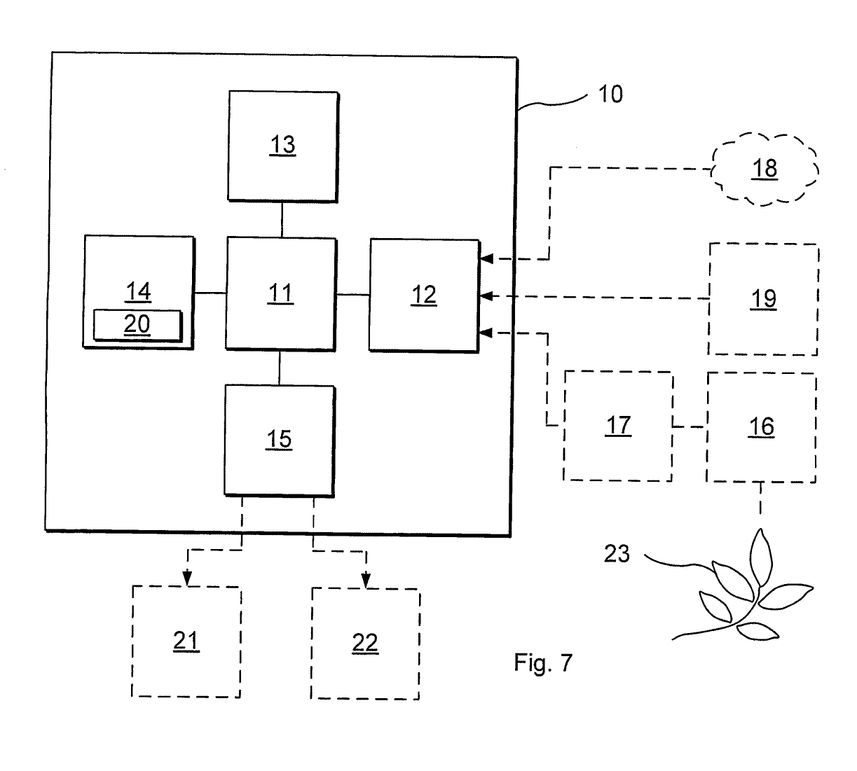

FIG. 7 shows a schematic diagram of an analyser.

The embodiment herein described illustrates principles of the invention carried out on a typical biological problem, here a problem from plant developmental physiology—a comparison of proteins isolated from three types of in vitro grown tissues of horseradish (Armoracia lapathifolia Gillib.) that differ in physiological conditions—leaves, tumour and teratoma.

All analysed tissues related to this biological problem (leaf, tumour and teratoma) are to be compared with regard to their protein expression patterns. All tissues were of the same genetic origin; tumours were induced on leaf fragments with Agrobacterium tumefaciens B6S3; teratoma, in the form of shoots with malformed leaves represented an unsuccessful way of tissue reorganization. A transition from one tissue pattern to another depends on modifications of gene expression; consequently changes in the proteome, a protein complement of the genome, should be visible in electrophoretic protein patterns.

In this embodiment of the invention in vitro grown horseradish (Armoracia lapathifolia Gillib.) leaves (L), tumour (T) and teratoma (Tr) tissue cultures were maintained on the solid MS nutrient medium without any growth regulator. Culture conditions were: 24° C., 16-h photoperiod and irradiation of 33 μmol m⁻² s⁻¹. Primary tumours had been induced on leaf fragments with a wild octopine strain B6S3 of Agrobacterium tumefaciens, according to Horsch et al. (Transgenic plants. Cold Spring Harb Symp Quant Biol 1985, 50, 433-437.) During sub-culturing two morphologically different tissue lines were established: one, unorganized tumour line (T) and the other, shoot-producing teratoma line (Tr).

Soluble proteins were extracted from tissues in the exponential phase of growth (12 days after subculturing). Tissue samples were homogenised in the ice cold 0.1 M Tris/HCl buffer (pH 8.0) containing 17.1% sucrose, 0.1% ascorbic acid and 0.1% cysteine/HCl. Tissue mass (g) to buffer volume (ml) ratio was 1:5 for leaves, 1:1.2 for teratoma and 1:0.9 for tumour tissue. The insoluble polyvinylpyrrolidone (cca 50 mg) was added to tissue samples before grinding. The homogenates were centrifuged for 15 min at 20 000×g and 4° C. The supernatants were ultracentrifuged for 90 min at 120 000×g and 4° C.

Protein content of supernatants was determined according to Bradford method using bovine serum albumin as a standard. Samples were denatured by heating for 3 min at 100° C. in 0.125 M Tris/HCl buffer (pH 6.8), containing 5% (v/v) β-mercaptoethanol and 2% (w/v) SDS (sodium dodecyl sulphate). For SDS-PAG-electrophoresis 12 μg of proteins per sample were loaded onto the gel.

As shown in FIG. 1 the first step 101 is the preparation of a number of chromatographic experiments in order to obtain measurements. In this case the chosen chromatographic method was SDS-PAG-electrophoresis. Of course mass spectrometric experiments could be used instead.

The SDS electrophoresis in 12% T (2.67% C) polyacrylamide gels, with buffer system of Laemmli (1970) was run in Biorad Protean II xi cell at 100 V for 45 minutes and at 220 V for a further four hours.

It is believed that a number of repeated measurements (3 as a minimum) is needed for each tissue type, and/or for each measurement condition (gel batch, position on a gel) that is suspected to cause systematic errors. Therefore in the example measurements were carried out on six samples from each of the tissue cultures (L, T and Tr) resulting in 12 gels in total. Protein bands were visualised by silver staining (Blum et al. 1987).

Each gel produces 4 columns (or “lanes”) for each of the three tissues (outer left, inner left, inner right and outer right). The gels were scanned on an Umax Astra 2200 scanner with the resolution set to 300 dpi. An extract from one of the scanned gels is shown in the centre of FIG. 5 showing three representative lanes of the 12. In FIG. 5, lane 1 is the leaf, lane 2 is the teratoma and lane 3 is the tumor.

To obtain the measurements of physiochemical attributes of the plurality of tissues with first, second and third physiological conditions in a computer readable format, i.e. in the form of a data set in a first attribute space, three line profiles of each lane (a part of the gel with separated proteins of one sample) were created using the UTHSCSA Image Tool 3.00 software and exported to text files at step 102 (FIG. 1). The start and the end of the lane to be analysed were manually fixed from one easily discernable protein band at the cathodic side and the other at the anodic side of the lane.

At this stage the data set comprises a large matrix with data representing the coloration intensity of each pixel along each of the three line profiles for each of the four gel positions of the six gels samples for each of the three tissue types i.e. a matrix with 216 rows representing the protein profiles and numerous columns representing the pixel number and each element of the matrix representing the coloration intensity of the respective pixel in the respective protein profile.

In order to reduce the number of columns in the matrix, the profiles were split into windows of the optimal size in step 103 (FIG. 1) using an overlapping windowing scheme and exposing each window size to an unsupervised and supervised test using the Weka 3-5-6 data mining suite.

Optimal window size is determined by forcing simultaneously high log-likelihood for the unsupervised test and high ratio of accuracy to number of overlapping windows in a supervised test as depicted in FIG. 2 which illustrates determining optimal floating window size. The x-axis shows the z parameter (reciprocal window size). The left y-axis and the associated curve (hollow squares, dotted line) show the log likelihood value reported by the EM clustering algorithm. The right y-axis and the curves drawn with black triangles and diamonds denote classification accuracy by tissue type for the SVM and kNN classifiers respectively. The vertical dotted line drawn at z=56 denotes the optimal window size determined by the highest accuracy achieved by the kNN classifier.

The unsupervised test was performed using expectation maximization algorithm, 100 times for each z with different random seeds. The highest average log likelihood ratio of 100 runs would indicate optimal z.

The supervised test was performed using the k nearest neighbour algorithm (kNN classifier), which was used to classify data by tissue using datasets with different z values; the optimal z being the one with the highest kappa statistic in 10 runs of tenfold cross-validation. These results were compared with the results obtained using SVM algorithm in the same fashion, as shown in FIG. 2.

Once optimal window size is determined, the individual measurements are binned into windows according to the optimal windowing scheme.

In this case the line profiles were split into overlapping windows of size 1/z, where length of overlaps was a half of the window size. The total number of windows per line profile was therefore 2z−1; for each window the arithmetic mean of pixel coloration intensities was computed. This procedure was necessary because of inevitable inconsistencies in the gel structure that cause areas in the profiles to seem slightly ‘compressed’ or ‘expanded’ in comparison with other samples. There are also slight variations in the total lane length making a pixel-by-pixel comparison infeasible. Smaller windows (larger z) preserve more information but make the method more sensitive to shifts as described above; larger windows (smaller z) are more robust but less informative. The parameter z was systematically varied from 16 to 256 in steps of 8 to find an optimal window size. We used overlapping windows instead of simply consecutive ones, because of the possibility that a relevant protein band can be positioned exactly over the window border. Because of the slight local shifts, the same band could sometimes be read as a part of one window and the other time as a part of the following window. In these cases, the overlapping windows would contain the band of interest.

After computation of mean window intensities, a median of corresponding windows in the three profiles for each lane was determined to lessen the influence of gel irregularities on the intensity scores, resulting in one floating-window profile with 2z−1 attributes per sample. The datasets were then standardized, so that the windows of a single sample had a mean of 0 and standard deviation of 1; this was done to decrease the influence of staining variation. The data sets, in this embodiment 72 protein profiles (24 replicas of each tissue), were labelled by (i) the tissue type (leaf, teratoma or tumour), (ii) the gel batch number (1-6) or (iii) by lane position on the gel (outer left, inner left, inner right or outer right).

A diagrammatic illustration of windowing is shown in FIG. 5, in the centre of which it can be seen that the gels are overlaid with windows numbered to the right of lane 1.

Having carried out windowing and computed the median of the three profiles per lane, the dataset is reduced to a more manageable size with 72 rows and the same number of columns as windows i.e. 111.

The fixed representation of the reduced dataset can be used to build a classification model at step 105 (FIG. 1) for future tissue type classification of unknown samples.

The reduced data set is then projected into a second attribute space using a projection technique such that the projected data is described as a plurality of components mathematically constructed from the original data set. In this example the projection technique used at step 104 (FIG. 1) is principal component analysis (PCA). PCA is a technique that creates linear combination of the original attributes, such that the new attributes are orthogonal and such that the greatest variance of the data lies along the first attribute (principal component), the second greatest variance on the second attribute, and so on. PCA can be performed by several methods including finding the eigenvectors of the covariance matrix of the matrix set, by performing singular value decomposition on the data set or by a Hebbian learning process.

However, it is believed that other projection techniques that create new attributes by combining, in a linear or non-linear fashion, the original attributes would work equally well. For example correspondence analysis, independent component analysis (ICA), linear discriminant analysis (LDA), kernel PCA, autoencoders and similar encoding/decoding methods based on the neural network paradigm, as well as filtering techniques such as discrete cosine transform, discrete Fourier transform and wavelet transform could be used instead.

An optional step (106a, FIG. 1) following the use of a projection technique and preceding the use of a feature selection method is discarding of components that are suspected to be derived from noise, judging by eigenvalues (i.e. the variance) reported by PCA, position in the frequency spectrum generated by a Fourier transform or a similar measure computed in an unsupervised manner, i.e. independently of physiological condition class assignment or known sources of systematic errors.

The first three columns in FIG. 3 (PC set, PC and % var) show that the first 13 principal components (PCs) contain 95% of the original variance in the data (i.e. 95% of the information in the matrix data set) cut down into 13 components. These 13 components thus describe the measured data in a significantly reduced form (13 components rather than 111 columns). The 5% which is not in the first 13 components would be related to noise and is therefore removed from further processing in step 106a (FIG. 1).

Next, in step 106b (FIG. 1), the data set which has undergone PCA is filtered in the second attribute space using a feature selection method to determine which components of the data set are most relevant for determining the different physiological conditions by comparing for each individual component, the distribution of values for that component relating to the first physiological condition, the distribution of values for that component relating to the second physiological condition and the distribution of values for that component relating to the third physiological condition and discarding those components where the difference between the distribution of values in respect of the first, second and third physiological conditions is low, to provide a filtered data set. In this embodiment the filtering is carried out using ReliefF as the feature selection method (see Robnik-\check{S}ikonja, M., Kononenko, I., Theoretical and Empirical Analysis of ReliefF and RReliefF. Machine Learning 53 (2003) 23-69). The ReliefF procedure was carried out based on each of the three labels in the data namely (i) the tissue type (leaf, teratoma or tumour), (ii) the gel batch number (1-6) and (iii) by lane position on the gel (outer left, inner left, inner right or outer right).

ReliefF operates on subsets of data chosen by a locality criterion; the neighbourhood size parameter was set to k=3. This heuristic approach quantifies an attribute's merit in context of possible non-linear interactions between attributes. This is in contrast to scoring each attribute without consideration of other attributes, as it is the case with ‘myopic’ measures like the Student's t-statistic. A single run of tenfold cross-validation in Weka Explorer module was employed to assess reliability, where in each iteration ReliefF was run on 9/10 of the dataset (class distribution was preserved), and average scores/rank as well as maximum deviations from average recorded.

Although in this embodiment ReliefF was the chosen feature selection method, other feature selection methods that evaluate relative importance of attributes could be applied in this invention. These include, but are not limited to: techniques based on conditional entropy measures (information gain, Chi-squared score, Gini index, and similar), techniques involving a program routine (wrapper) that performs a number of classification or regression experiments involving a supervised machine learning method where one or a set of attributes are left out in each experiment, or other feature selection methods operating on local class boundaries, as exemplified in the Relief method family adapted to noisy, incomplete data sets and/or data sets with mutually dependent features.

The fourth to sixth columns headed “merit” show the ReliefF scores of each of the 13 principal components based on each of the labels, where each full 0.05 in the score equals one dot, and each full 0.025 equals half a dot. The most important scores from the point of view of the invention are the scores in the “tis” (tissue type) column as these show which of the principal components correlate most strongly with the different tissue types (i.e. have value distributions that show the biggest difference based on the different “tissue” labels). Thus it can be seen that the three principal components with the most relevant data for distinguishing between tissue types are principal components 1, 6 and 7 (which have the highest number of dots in the “tis” column).

On the other hand, although principal component 2 contains the second largest amount of data (12.8% var) the data it contains is not useful for distinguishing the tissue type and principal components 3, 4 and 5 appear to include data which is more related to systematic errors induced by the differences between gels used rather than the type of tissue.

FIG. 4 illustrates diagrammatically the results of the ReliefF scoring i.e. that the first and sixth components contain much more useful information for distinguishing between tissue types than the first and second principal components, despite the fact that there is more information in the first and second components. In the upper graph first and second principal components of the data are visualized, displaying ˜63% of the original information. This graph shows that separation of untransformed (leaf) and transformed (teratoma and tumour) tissues is possible based on these two components. On the other hand, the lower graph which is a visualization of PC1 vs. PC6 allows for easy separation of all three tissue types, despite containing less information: ˜53%.

Accordingly in this embodiment at step 106b (FIG. 1) those components where the difference between the distribution of values in respect of the first, second and third physiological conditions is low are all the components except PCs 1, 6 and 7. Therefore all of the remaining principal components are discarded to provide a filtered data set including only the components that are most relevant for determining the different physiological conditions of the samples in the data.

The next step 107 (FIG. 1) is back projecting the filtered data set back to the first attribute space using a reversion of the projection technique that was used in step 104 (FIG. 1) (PCA). The results of this back-projection, labelled 108 in FIG. 1 can be visualised (step 110, FIG. 1) and are shown as an artificial gel in the column of FIG. 3 labelled “only PCs in set” and the row labelled “tissue”. An enlarged view of this “artificial gel” is shown in FIG. 6.

FIG. 3 also depicts back-projected data sets for other target classifications, showing those that relate to the gel batch, those that cannot be correlated and a back projection of all of components 1-13; this information is not relevant for the present invention, but may be of academic interest.

Also of academic interest may be the back-projected data sets under the heading “PCs 1-13 not in set”. These show the back projection of the principal components filtered out of the sets to their left, i.e. in the row labelled tissue where the set comprises PC's 1, 6 and 7, PCs 2-5 and 8-13 are shown. Classification accuracy in relation to all of the data in FIG. 3 is expressed as the kappa statistic estimated using 10 runs of 10-fold cross-validation, obtained with Support Vector Machines classifier.

Although there is a greater contrast between the three lanes in the back-projected artificial gels shown in FIG. 6, it is still not obvious to the human eye which regions of the back projected data (i.e. what fractions of the biological mixtures) are most important for distinguishing between the teratoma (lane 2) and the tumour (lane 3).

However, in step 109 (FIG. 1) the back-projected data set is filtered in the first attribute space using a feature selection method to determine which attributes of the back-projected data set are most relevant for determining the different physiological conditions by comparing how the distribution of values of each attribute of the data set differs between the first physiological condition and the second physiological condition and discarding those attributes where the difference in distribution of values is low. The feature selection method employed for this step was the ReliefF ranking scheme. Side charts showing the results of this filtering step are shown in FIG. 5. Bar heights in side-charts show window merits (ReliefF scores) for discrimination of leaf tissue vs. teratoma and tumour (left hand side chart), or teratoma vs. tumour (right hand side chart). In order to illustrate the improvement of the invention, the raw data was also filtered using ReliefF and this comparative data is represented by the black bars, whereas the white bars show the ReliefF scores for the filtered data, with only PCs 1, 6 and 7 retained.

It can be seen that for determining the most important fractions to distinguish leaf from the transformed tissues (teratoma and tumour) (left-hand side chart), the white bars are not a great deal taller than the black bars. This indicates that for distinguishing between these samples (which are relatively different physiologically and physiochemically) the method has not been exceptionally useful, although it has revealed that the fractions in the region of window 60 are important which could warrant further scientific investigation.

On the other hand, it can be seen that in order to determine the most important fractions that distinguish the teratoma from the tumour (a more complex problem in view of the greater similarity between these physiological conditions and one where visual inspection of the gels reveals no characteristic patterns) the method of the invention has strongly improved the results. The average ReliefF score of the top 20 windows in the filtered back-projected data is 0.339 compared to 0.115 in the raw data and the height of the white bars is clearly much greater than that of the black bars.

The three plots at the right hand side of FIG. 5 show distributions in the values of three windows that have shown largest increases in importance after filtering; crosses are teratoma samples, and circles are tumour samples; two leftmost columns are raw data, and two rightmost columns the filtered data.

Having identified that these windows are most important, the proteins in these windows could be isolated from the gel and further tests carried out.

Alternatively, if for example the biological mixtures that had been studied were two different types of cancer with different physiological conditions, one of which reacted to a drug, the other of which did not, but which were undistinguishable otherwise, having identified the most important fractions to distinguish between them, it would be possible to build a reliable model to discriminate between the classes (step 111, FIG. 1). It will be understood that once such a model was produced it would be possible to determine which class of cancer a particular patient has and tailor the drug regime accordingly i.e. not administer a drug which would be of no harm to the cancer, but only cause side effects.

Referring to FIG. 7, the analyser 10 which carries out the steps mentioned above will now be described in terms of functional or logical components. It will be appreciated that some of the components could be combined to provide the same overall functionality if required.

The analyser 10 includes a controller 11, an input 12, a computation engine 13, storage 14 and an output 15. The controller 11 controls overall operation of the analyser 10.

The input 12 obtains measurements of physiochemical attributes for cells or tissues. In the abovementioned description, the measurements of data relating to biological mixtures 23 are obtained from a measurement device 16 and scanner 17; the measurement device 16 consists of a Biorad Protean II xi cell. It could alternatively be another chromatographic instrument or a mass spectrometer, displaying measurements as an image which can be scanned by scanner 17. However, the measurement device 16 could equally output the measurements directly to the analyser, or could form part of the analyser 10.

In this case, if the measurement device is chromatographic it would include: a mobile phase supply system; a sampling system arranged to receive the biological mixtures 23 comprising first cells or tissues with first physiological conditions and second cells or tissues with second, different, physiological conditions; a stationary phase system; and

a detector arranged to detect the quantity of different fractions; whereby, measurements of physiochemical attributes of first cells or tissues with first physiological conditions and second cells or tissues with second physiological conditions, in the form of a first data set in a first attribute space are obtained from the detector, either by way of an output into the input 12 or by a direct feed to the controller 11.

Alternatively, if the measurement device comprises a mass spectrometer connected to the analyser 10, the results of the spectrometric detection would be outputted via an output in the mass spectrometer to the input 12. If mass spectrometer forms part of the analyser 10, the results of the mass spectrometric detection could simply be fed directly to the controller 11.

As an alternative to inputting the measurements of physiochemical attributes to the analyser straight from the measurement device, the measurements could be stored and then obtained from a network 18, for example as an e-mail attachment or download, or from a data transfer device 19 such as a CD or USB mass storage device.

The computation engine 13 performs mathematical operations such as the feature selection method and projection techniques on the data sets in the first and second attribute spaces.

The storage 14 typically comprises a non-volatile memory such as an internal or external hard disk drive. The measurement information obtained by the input 12 can be written to the storage 14 for archiving if desired. A computer program 20 is stored in the storage 14 which, when executed, causes the analyser 10 to operate under the control of the controller 11. The computer program 20 may be received via the input 12, for example in a signal from the network 18 or as an executable file from a data transfer device 19.

The output 15 enables information processed by the analyser to be used by other entities and/or to be provided to an operator. For example, the analyser 10 can be connected to a printer 21 and/or a display 22.

1. An analyser for determining relative importance of fractions in biological mixtures separated by a chromatographic or mass spectrometric method originating from cells or tissues with different physiological conditions, the analyser arranged to:
a. obtain measurements of physiochemical attributes of first cells or tissues with first physiological conditions and second cells or tissues with second physiological conditions, in the form of a first data set in a first attribute space;
b. project the data set into a second attribute space using a projection technique such that the projected data set is described as a plurality of components mathematically constructed from the first data set;
c. filter the data set in the second attribute space using a feature selection method to determine which components of the data set are most relevant for determining the different physiological conditions by comparing for each individual component, the distribution of values for that component relating to the first physiological condition and the distribution of values for that component relating to the second physiological condition and discarding those components where the difference between the distribution of values in respect of the first and second physiological conditions is low, to provide a filtered data set;
d. back-project the filtered data set back to the first attribute space using a reversion of the projection technique used previously at step (b) to provide a back-projected data set; then
e. filter the back-projected data set in the first attribute space using a feature selection method to determine which attributes of the back-projected data set are most relevant for determining the different physiological conditions by comparing how the distribution of values of each attribute of the data set differs between the first physiological condition and the second physiological condition and discarding those attributes where the difference in distribution of values is low; and
f. output the results of step (e) in a human-readable format such that the physiochemical attributes that correspond to the differences in physiological conditions between the plurality of cells or tissues can be identified.

2. An analyser according to claim 1 arranged to obtain measurements in the form of a first data set in a first attribute space by creating line profiles from an image displaying the results of a chromatographic or mass spectrographic method.

3. An analyser according to claim 1 further comprising:
a mobile phase supply system;
a sampling system arranged to receive the biological mixtures comprising first cells or tissues with first physiological conditions and second cells or tissues with second, different, physiological conditions;
a stationary phase system; and
a detector arranged to detect the quantity of different fractions; whereby,
measurements of physiochemical attributes of first cells or tissues with first physiological conditions and second cells or tissues with second physiological conditions, in the form of a first data set in a first attribute space are obtained from the detector.

4. An analyser according to claim 3 wherein the mobile phase supply system, sampling system, stationary phase system and detector are components of an electrophoresis instrument.

5. An analyser according to claim 1 further comprising a mass spectrometer including a detector arranged to detect fractions of biological mixtures; whereby,
measurements of physiochemical attributes of first cells or tissues with first physiological conditions and second cells or tissues with second physiological conditions, in the form of a first data set in a first attribute space are obtained from the detector.

6. An analyser according to claim 1, comprising an input arranged to carry out step (a), a computation engine arranged to carry out steps (b to (e) and an output arranged to carry out step (f).

7. A method of determining relative importance of fractions in biological mixtures separated by a chromatographic or mass spectrometric method originating from cells or tissues with different physiological conditions, comprising:
a. obtaining measurements of physiochemical attributes of first cells or tissues with first physiological conditions and second cells or tissues with second physiological conditions, in the form of a first data set in a first attribute space;
b. projecting the data set into a second attribute space using a projection technique such that the projected data set is described as a plurality of components mathematically constructed from the first data set; and characterised by:
c. filtering the data set in the second attribute space using a feature selection method to determine which components of the data set are most relevant for determining the different physiological conditions by comparing for each individual component, the distribution of values for that component relating to the first physiological condition and the distribution of values for that component relating to the second physiological condition and discarding those components where the difference between the distribution of values in respect of the first and second physiological conditions is low, to provide a filtered data set;
d. back-projecting the filtered data set back to the first attribute space using a reversion of the projection technique used previously at step (b) to provide a back-projected data set; then
e. filtering the back-projected data set in the first attribute space using a feature selection method to determine which attributes of the back-projected data set are most relevant for determining the different physiological conditions by comparing how the distribution of values of each attribute of the data set differs between the first physiological condition and the second physiological condition and discarding those attributes where the difference in distribution of values is low; and
f. outputting the results of step (e) in a human-readable format such that the physiochemical attributes that correspond to the differences in physiological conditions between the plurality of cells or tissues can be identified.

8. The method of claim 7 wherein the chromatographic or mass spectrometric method is capillary electrophoresis, gel electrophoresis, paper electrophoresis, ion-exchange chromatography, affinity chromatography, gel filtration, partition chromatography, or adsorption chromatography.

9. The method of claim 8 wherein the chromatographic method is gel electrophoresis.

10. The method of claim 7 wherein the chromatographic or mass spectrometric method is mass spectrometry.

11. The method of claim 7, wherein the measurements of physiochemical attributes of first cells or tissues with first physiological conditions and second cells or tissues with second physiological conditions obtained at step (a) are grouped into windows having positions, lengths and overlaps adjusted to optimize a score representative of the relevance and/or consistency of the data set.

12. The method according to claim 11, wherein the score used as optimization criterion comprises a data distribution measure derived from applying a statistical method to the data set.

13. The method according to claim 11, wherein the score used as optimization criterion comprises a data distribution measure derived from applying an unsupervised machine learning method to the data.

14. The method according to claim 11, wherein the score used as optimization criterion comprises an error measure reported by a supervised machine method applied to the data attempting to discriminate between the physiological conditions of cells or tissues used to produce the data set.

15. The method according to claim 7, wherein the projection technique is principal component analysis, independent component analysis, linear discriminant analysis, or kernel principal component analysis.

16. The method according to claim 15 wherein the projection technique is principal component analysis.

17. The method according to claim 7, wherein the projection technique is an autoencoder or like encoding/decoding method based on the neural network paradigm.

18. The method according to claim 7, wherein the projection technique is discrete cosine transform, discrete Fourier transform or a wavelet transform technique.

19. The method according to claim 7, further comprising discarding components that are suspected to be derived from noise after the projection step (b).

20. The method according to claim 7, wherein the feature selection method of either or both of steps (c) and (e) comprises a technique based on conditional entropy measures.

21. The method according to claim 7, wherein the feature selection method of either or both of steps (c) and (e) comprises a technique based on a program routine that performs a number of classification or regression experiments involving a supervised machine learning method, where one or a set of attributes are left out in each experiment.

22. The method according to claim 7, where wherein the feature selection method of either or both of steps (c) and (e) comprises a technique operating on local class boundaries, such as the Relief family of methods.

23. The method of claim 22 wherein the feature selection method of either of steps (c) and (e) comprises the ReliefF method.

24. The method of claim 22 wherein the feature selection method of both of steps (c) and (e) comprises the ReliefF method.

25. The method of claim 7, wherein the measurements of physiochemical attributes of first cells or tissues with first physiological conditions and second cells or tissues with second physiological conditions are repeated at least three times for each of the first and second cells or tissues and the results of the at least three measurements are all included in the first data set in the first attribute space.

26. A computer program comprising instructions which, when executed, cause an analyser to perform the method of claim 7.

27. A computer-readable medium comprising a computer program according to claim 26.

28. A signal carrying the computer program according to claim 26.
